(19) United States
(12) Patent Application Publication
(10) Pub. No.: US 2011/0028974 A1
(43) Pub. Date: Feb. 3, 2011

(54) DEVICE FOR REDUCING A BONE FRACTURE

(76) Inventor: Antonio Chemello, Udine (IT)

Correspondence Address: BIRCH STEWART KOLASCH & BIRCH, PO BOX 747, FALLS CHURCH, VA, 22040-0747, US

(21) Appl. No.: 12/867,297

(22) PCT Filed: Feb. 19, 2009
(86) PCT No.: PCT/IB09/00320

§ 371 Date: Aug. 12, 2010

(30) Foreign Application Priority Data
Feb. 27, 2008 (IT) VI2008A000046

(51) Int. Cl. A61B 17/58

(52) U.S. Cl. 606/63

(57) ABSTRACT

The present invention relates to a device for reducing a bone fracture (1, 100, 200, 300, 400, 500, 600, 700) comprising a main body (2) and one or more anchoring elements (3) suitable for being moved by actuating means (4) from a position at rest inside the main body (2) to a working position outside the main body (2). Said device also comprises elastic means that cooperate with said anchoring elements (3) to push said anchoring elements (3) outside the main body (2) through corresponding openings (5) when the actuating means (4) are operated.

The invention relates to orthopedic and traumatological surgery.

In particular, the invention concerns the surgical treatment of bone fractures.

More in detail, the invention relates to a device for reducing a bone fracture, also called an intramedullary nail, with improved functional characteristics by comparison with the nails of known type, for use in reducing bone fractures, and long bone fractures in particular.

It is common knowledge that surgery is a widely-used method for treating bone fractures, especially those involving the long bones. Surgery has ample means of synthesis available for reducing such fractures, including the intramedullary nail, which is being used more and more.

This device or so-called nail, for reducing a bone fracture comprises an elongated tubular body that is inserted inside the suitably-milled medullary canal of the fractured bone.

According to a known embodiment, the intramedullary nail comprises a set of anchoring elements, also called pins, contained inside the tubular body. These pins are designed to emerge through suitable openings provided on the tubular body in order to become implanted in the cortical part of the fractured bone, enabling the distal fixing of the nail to the bone.

The emergence of the pins is actuated by the surgeon with the aid of suitable tools, which are used to take action on actuating means comprising mechanical members inserted in the body of the nail.

At the end of the period of convalescence, the surgeon takes action again, using the above-mentioned tools, to make the pins withdraw completely inside the tubular body, enabling the device to be removed from the bone.

A first drawback of this type of device lies in that the procedures involved in making the pins emerge and withdraw are not safe and reliable.

More in particular, a drawback of this type of nail lies in that the aforesaid pins become caught against the nail during the installation and extraction of the nail.

This interferes, moreover, with the proper emergence and optimal withdrawal of the pins from/in the tubular body.

Another disadvantage lies in that the surgeon may be unable to make all the pins withdraw and some of them impact against the edge of the above-mentioned opening or seize up.

Another drawback, that is related to those mentioned above, lies in that a considerable effort is required on the part of the surgeon to govern the emergence and withdrawal of the pins.

A further drawback lies in that said effort may lead to the rupture of some of the pins, with the consequence that some of the resulting fragments remain inside the bone, possibly giving rise to clinical complications.

Another drawback relating to those previously mentioned lies in that surgeons are unable to take action in full control and in safe conditions, and they cannot be assured the sensitivity they need in the procedure to accurately control the position reached by the pins inside the bone.

The object of the present invention is to overcome the above-mentioned drawbacks.

A particular object of the invention is to realise a device for reducing bone fractures with improved functional features by comparison with those of the nails of known type, for use in reducing fractures of long bones, that enables the above-mentioned drawbacks to be overcome.

Another object is to realise a device suitable for being fixed distally to the cortical part of the fractured bone in a rapid and reliable manner

Another object is to realise a device in which the procedure for withdrawing the pins inside the tubular body is rapid and reliable.

Another object is to realise a device in which any seizure of the pins is avoided during the procedure for extending them from or withdrawing them into the tubular body.

Another object is to realise a device that is more straightforward to implant and remove than comparable nails of known type.

Another object is to realise a device that facilitates the surgeon's work during the procedures for the installation and removal of the device, and particularly that gives the surgeon greater control over its positioning and fixing to the bone than with comparable nails of known type.

Another object is to realise a device in which the pins complete the above-described steps smoothly, without encountering any obstacles or any other problem that might prevent or restrict said action.

Another object is to realise a device in which the trajectories covered by the pins in order to emerge from the nail and become anchored to the cortical part of the bone, and subsequently to withdraw therefrom, are controlled and improved by comparison with the devices of known type. More in particular, an object is to ensure that the trajectories covered by the pins during their emergence and withdrawal coincide substantially with one another.

A last but not least object is to realise a device that is easy to manufacture and assemble, economical, and user-friendly, in which the procedures involved in fixing it distally to the bone and later withdrawing the pins used to do so are particularly straightforward and require no particular effort on the part of the surgeon.

The above-mentioned objects are achieved by a device for reducing a bone fracture as described and characterised in the main claim.

Advantageous embodiments form the object of the dependent claims.

The proposed solution advantageously enables a device to be realised in which the system that achieves its distal fixing to the bone by means of the emergence of the pins and their subsequent withdrawal into their respective seats is more reliable and safe than in the devices of known type.

The proposed solution equally advantageously enables a device to be realised that assures the surgeon the ability to achieve the emergence and withdrawal of said pins easily and smoothly, in a calibrated manner and with a greater sensitivity. This advantageously enables any need to use x-rays to identify the position of the pins to be reduced to a minimum, thereby safeguarding the patient's health.

The proposed solution equally advantageously enables a device to be realised in which the emergence and withdrawal of the pins takes place slidingly and with considerably less effort on the part of the surgeon than with comparable nails of known type. This also enables any risk of the rupture of said pins and the dispersion of the resulting fragments inside the bone to be avoided.

The above-mentioned objects and advantages are better illustrated in the description of several preferred embodiments of the invention, given here as non-limiting examples, with reference to the attached drawings, wherein:

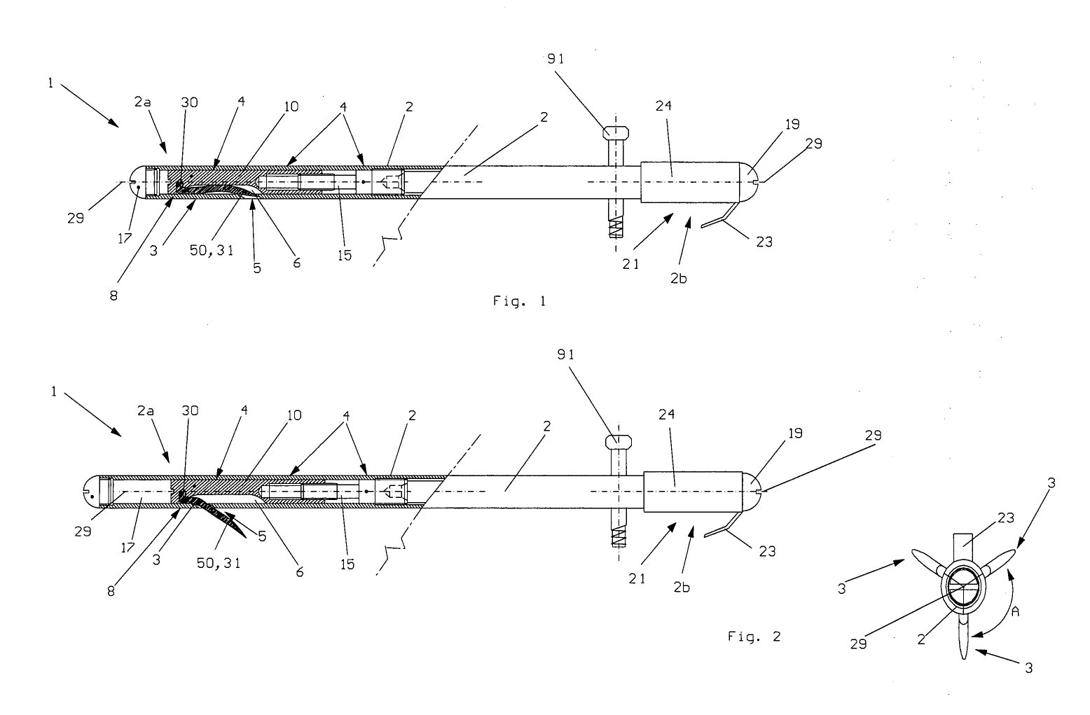

FIG. 1 shows a partial longitudinal sectional view of an embodiment of a the device according to the invention in a first working position;

FIG. 1a shows an exploded view of a longitudinal section of the device in FIG. 1;

FIG. 2 shows a partial longitudinal sectional view of the device in FIG. 1 in another working position, combined with a view thereof from above;

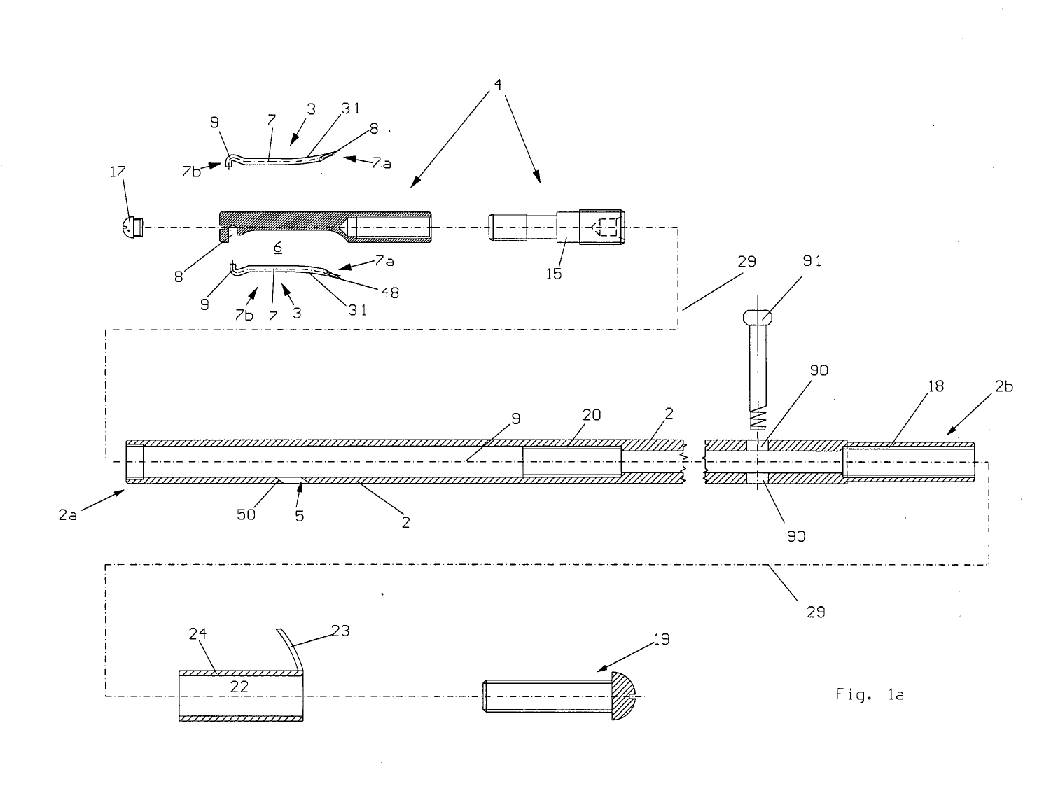

FIG. 3 shows a side view of an element of the nail in FIG. 1;

FIG. 4 shows another side view of the element in FIG. 3;

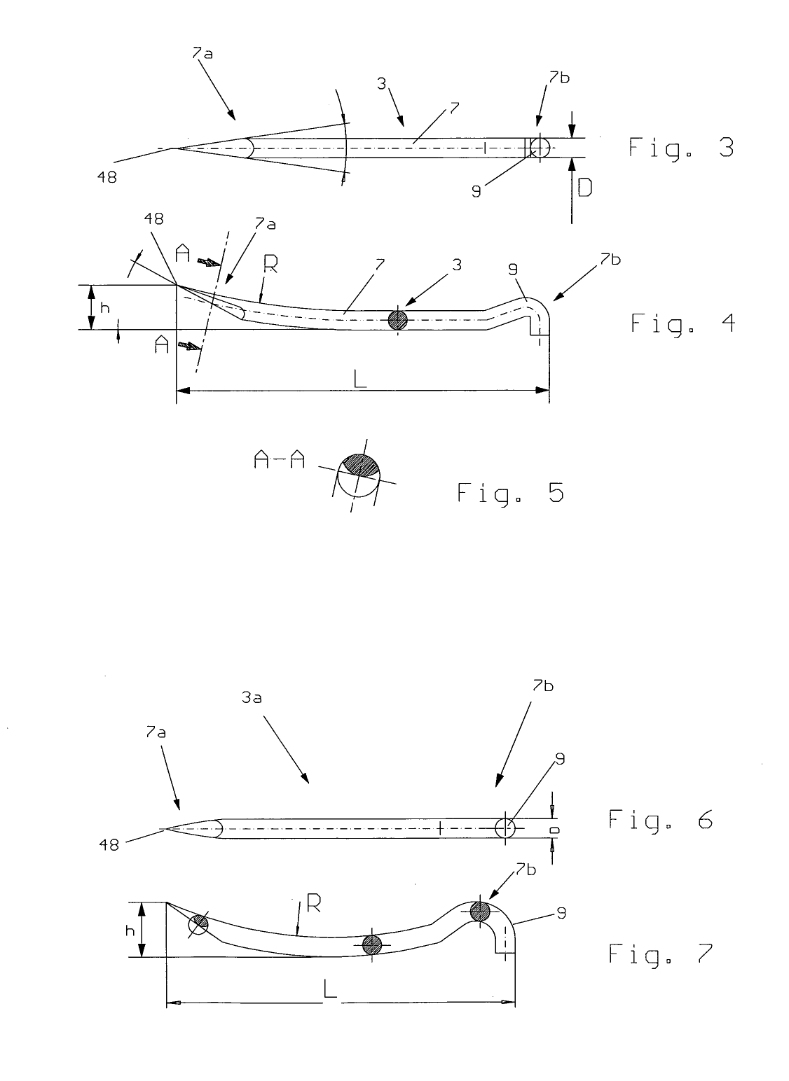

FIG. 5 shows a cross-sectional view of the element in FIG. 3;

FIG. 6 shows a side view of a variant of the element in FIG. 3;

FIG. 7 shows another side view of the element in FIG. 6;

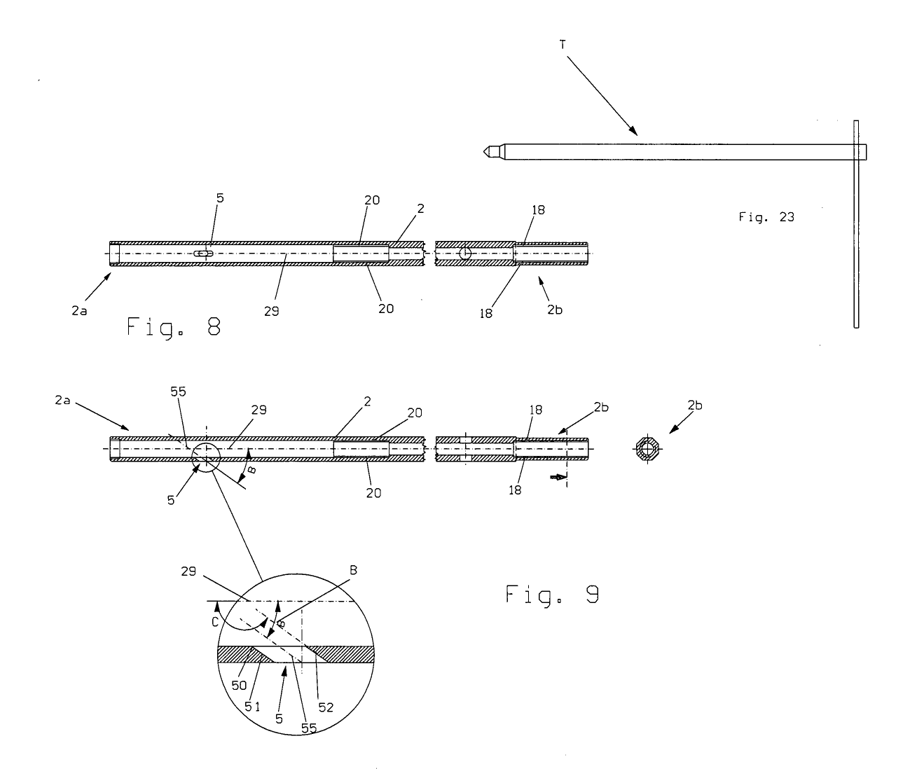

FIG. 8 shows a longitudinal sectional view of another element of the nail in FIG. 1;

FIG. 9 shows a further longitudinal sectional view and a cross-sectional view of the element in FIG. 8;

FIG. 10 shows a longitudinal sectional view of another element of the nail in FIG. 1;

FIG. 11 shows a cross-sectional view along Z-Z of the element in FIG. 10;

FIG. 12 shows a side view of another element of the nail in FIG. 1;

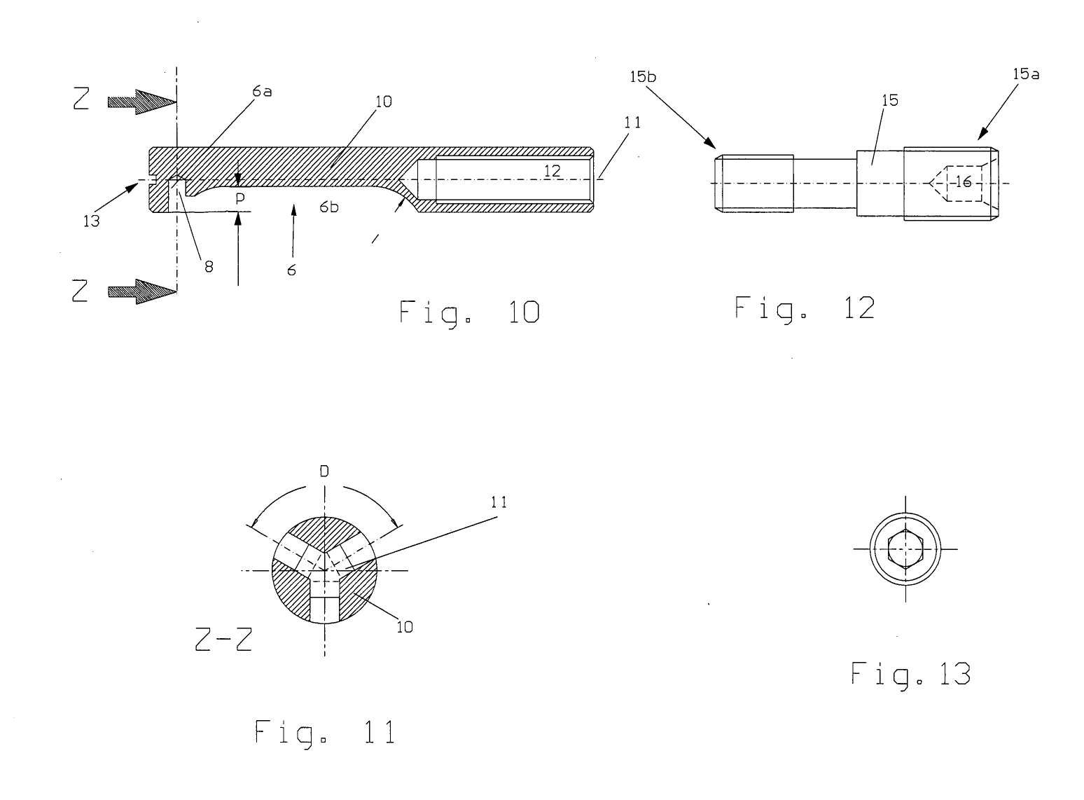

FIG. 13 shows a view from above of the element in FIG. 12;

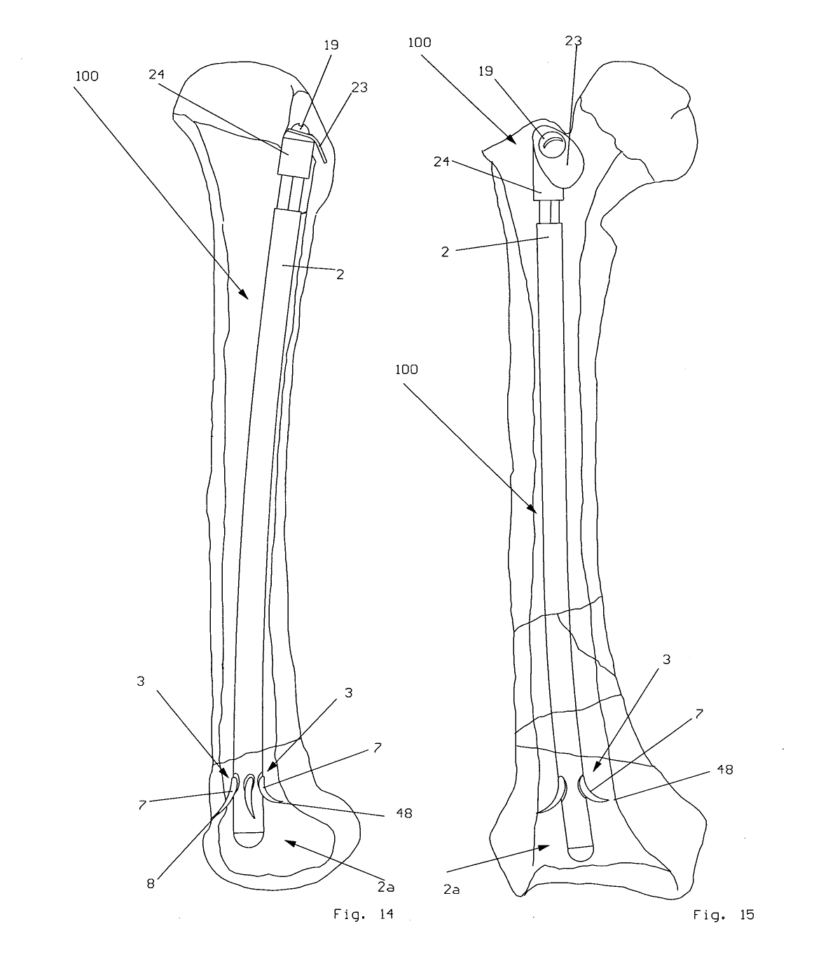

FIG. 14 shows a side view of another embodiment of the device according to the invention, applied to a bone with a simple fracture;

FIG. 15 shows a side view of the device in FIG. 14 applied to a bone with a multifragmentary fracture;

FIGS. 16 and 17 show two partial longitudinal sectional views of two further embodiments of a device according to the invention;

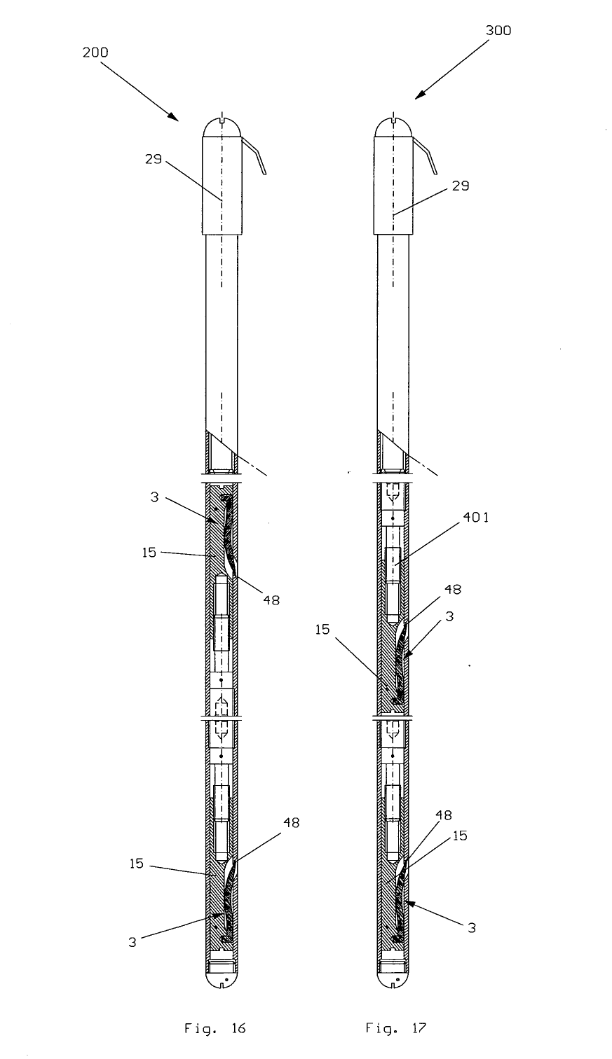

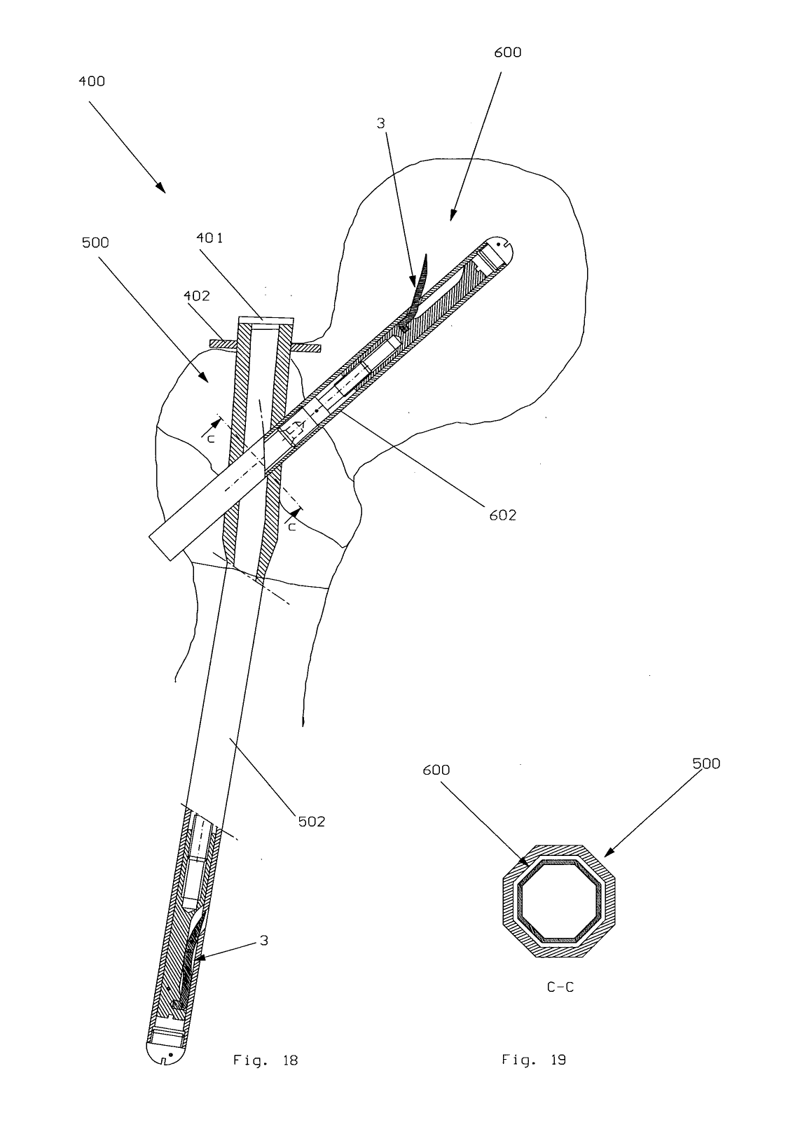

FIG. 18 shows a longitudinal sectional view of another embodiment of the device according to the invention, applied to a bone with a peri-/sub-trochanteric fracture:

FIG. 19 shows a cross-sectional view along C-C of the device in FIG. 18;

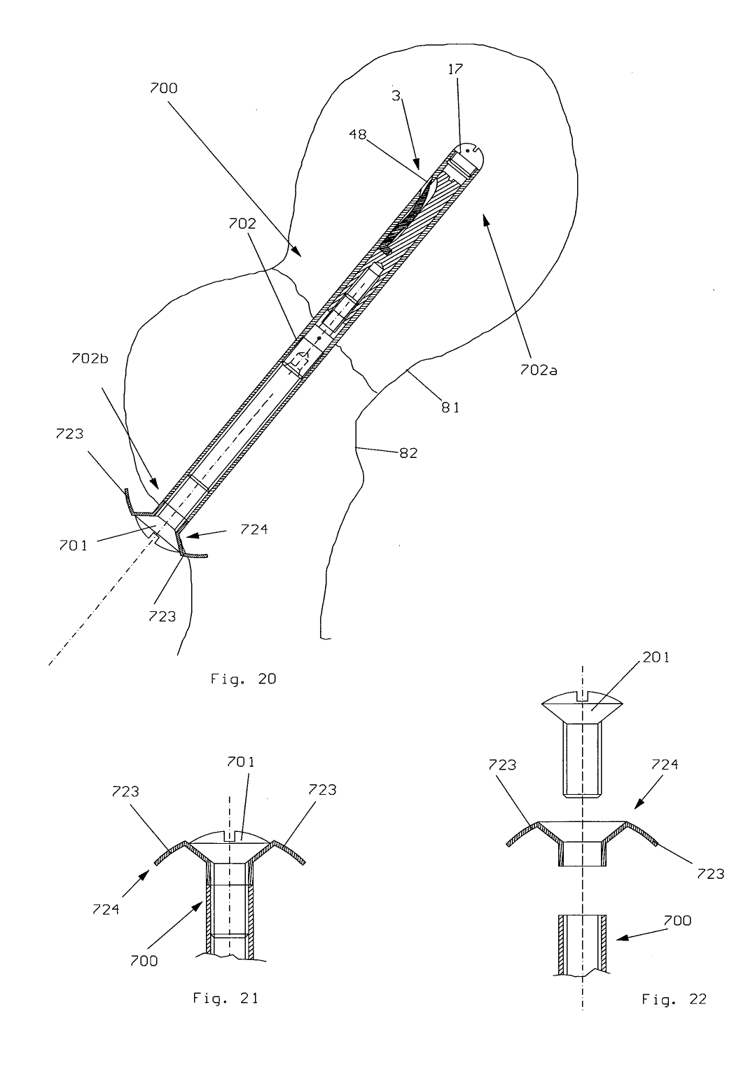

FIG. 20 shows a longitudinal sectional view of another embodiment of the device according to the invention, applied to a fracture of the neck of femur;

FIG. 21 shows a longitudinal sectional view of several parts of the device in FIG. 20;

FIG. 22 shows an exploded longitudinal sectional view of the parts shown in FIG. 21;

FIG. 23 shows a side view of the key used by the surgeon to take action on the device according to the invention;

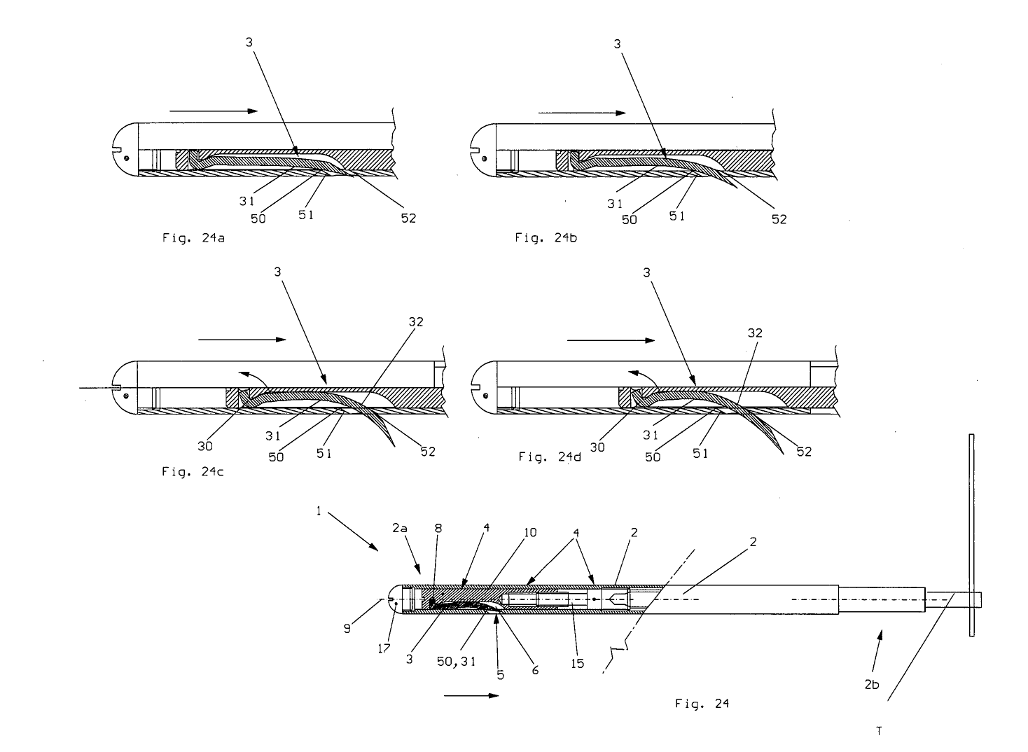

FIG. 24 shows a longitudinal sectional view of the device in FIG. 1, to which the key in FIG. 23 is coupled during an installation step;

FIGS. 24a to 24d each show an enlarged view of a partial longitudinal sectional view of several parts of the device in FIG. 1 in the various positions that they can occupy.

First of all, it should be noted that corresponding elements of the various embodiments are identified by means of the same numerical references.

The position indicators mentioned in the various embodiments can be logically transferred to the new position, in the event of any change in position.

While the following description and the related figures illustrate certain particular embodiments of the present invention, this is on the understanding that the present invention is not limited to said particular embodiments, but rather that the particular embodiments described herein merely clarify certain aspects of the present invention, the object and scope of which are defined in the claims.

The embodiments of the invention described below refer to a device for reducing bone fracture, and more in particular to an improved intramedullary nail suitable for use in reducing bone fractures, and especially those of the long bones.

In particular, said device is preferably for use in reducing fractures of bones such as the humerus, tibia, femur, or intratrochanteric or peri-/sub-tronchanteric fractures, and those affecting the neck of femur.

Clearly, the device can also be used to treat other types of fracture that necessitate the implantation of nails serving a similar functional purpose to that of the device according to the invention.

More precisely, the device according to the invention is designed to be implanted inside the suitably-milled medullary canal of a fractured bone.

A preferred, non-limiting embodiment of the device for reducing a bone fracture according to the present invention is shown in FIGS. 1, 1a and 2, where it has been globally indicated by the numeral 1.

It comprises a main body 2 and one or more anchoring elements 3, which are designed to be displaced using actuating means, indicated globally by the numeral 4, from a position at rest inside the main body 2, shown in particular in FIG. 1, to a working position, outside the main body 2, shown in FIG. 2.

In their position at rest, the anchoring elements 3 are contained in corresponding seats 6, visible in FIGS. 1, 1a and 2, from where they emerge through openings 5 in the main body 2.

It is emphasised that, in the preferred but non-limiting embodiment of the invention illustrated herein, the anchoring elements 3 are employed in a number amounting to three and they are arranged radially, at an angle A corresponding to 120° from each other, around the longitudinal axis 9.

This is on the understanding, however, that the number of anchoring elements 3 may differ in other versions.

According to the invention, the device 1 also comprises elastic means that cooperate with the anchoring elements 3 to drive said anchoring elements 3 towards the outside of the main body 2 when the actuating means 4 are operated.

In the preferred, non-limiting embodiment illustrated herein, the elastic means consist of the anchoring elements 3 themselves, also called pins, that are made in this case of an elastic and/or shape-memory material.

More in particular, the anchoring elements 3, shown in figures from 3 to 5, comprise a shank 7 with a first end 7a and a second end 7b, and with means 9 for connecting them to the actuating means 4.

The shank 7 is shaped and has a length L and a height h.

The cross-section of the shank 7 is preferably circular, with a diameter D.

In the preferred, non-limiting embodiment illustrated herein, the shaped shank 7 is arched and substantially reproduces an arc of a circle. In other embodiments, the shank 7 substantially reproduces an arc of an ellipse or of a parabola.

The first end 7a terminates with a pointed terminal portion 48, or tip, suitable for being placed in contact with the inside wall of the bone and becoming implanted therein. The pointed terminal portion 48 is obtained by removing a part of the end 7a and particularly through the intersection of two spheres.

Clearly, the shape of the shank 7, and the radius of curvature R in particular, together with the features of the material of which the shank is made, determine the elastic characteristics of each element 3.

The above-mentioned elastic action is achieved when the elements 3, also called pins, are displaced from their position at rest into their working position.

Said action is such that each anchoring element 3 advantageously comes into contact with the bone as soon as possible.

Moreover, said elastic action means that each anchoring element 3 can acquire different conformations or shapes.

More in particular, each anchoring element 3 can acquire at least a first conformation or shape when it is in its resting position inside its seat 6, and at least a second conformation or shape.

FIG. 1 shows a first conformation or shape that can be acquired by each element 3 when it occupies its a position at rest inside its seat 6.

It should be noted that each anchoring element 3 is forced into this first conformation, being compressed inside its corresponding seat 6. In fact, the edge 50 of the opening 5 interferes with the surface 31 of the element 3 that is consequently forced to become elastically deformed.

Each element 3 is arranged, moreover, so that its end 8 designed to engage in the bone O coincides with the corresponding opening 5 and does not extend from the main body 2 of the nail 1.

This advantageously enables a proper emergence of each anchoring element 3 to be guaranteed during the installation of the nail 1, preventing the element 3 from becoming caught in its corresponding seat 6, and thereby facilitating the implantation of each element 3 in the cortical tissue of the bone.

FIG. 2 shows the second conformation or shape acquired by each element 3 when it is in its working position.

It should be noted, moreover, that in the particular embodiment shown herein, the same above-described elastic action is also exploited when the actuating means are used to bring the elements 3 from their working position back to their position at rest.

The connecting elements 9 consist of the second end 7b of each pin 3, which is suitably shaped to fit inside a corresponding cavity 8 visible in FIGS. 1, 1a and 2, belonging to the aforesaid actuating means 4.

Said elements 9 enable each pin 3 to rotate around an axis 30 that is substantially orthogonal to the longitudinal axis 29 of the device 1, advantageously facilitating their radial emergence through the opening 5, as also described later on.

In the preferred but non-limiting embodiment of the invention discussed herein, the pins 3 have a global length L coming between 22 mm and 36 mm, and their end 7a has a radius of curvature R coming between 55 mm and 83 mm, and preferably amounting to 55.6 mm or 60 mm, or 66.5 mm, or 75 mm or 82.5 mm, while the diameter D of the shank 7 comes between 1.5 mm and 2 mm.

More in particular, in a preferred embodiment, the pins 3 have an overall length of 26.5 mm, their ends 7a have an angle of 25°, and the diameter of the main body 2 is 1.8 mm.

Clearly, the above-stated dimensions may need to differ in other embodiments of the present invention.

It should also be noted that the pins 3 are preferably made of a material that is compatible with the human body and that is elastically deformable.

More precisely, according to a preferred embodiment, the pins are obtained by a process of drawing and/or moulding of metal materials.

In particular, in the specific embodiment illustrated herein, the pins 3 are made using materials belonging to the family of stainless steels type AISI 316L.

Clearly, however, other types of material, even non-metallic, that have similar features to those of the above-specified materials, e.g. ceramics or plastics, or sintered materials, can also be used to manufacture the pins 3.

By way of example, FIGS. 6 and 7 show a different embodiment of an anchoring element, globally indicated by the numeral 3a, with a different shape.

In the non-limiting embodiment illustrated herein, the main body 2 comprises a hollow body with a first and a second end, respectively indicated in FIGS. 8 and 9 by the numerals 2a and 2b. More in particular, said hollow body comprises a tubular member with a preferably circular cross-section complete with the aforesaid through openings 5 around its lateral wall.

In the non-limiting embodiment illustrated herein, the main body also has a stretch at the upper end 2b that is substantially octagonal in shape externally, while the opposite, lower end 2a can be closed with a threaded plug 17, visible in FIGS. 1, 1a and 2.

Said plug 17 is designed to serve as a limit stop and to stop any sliding of the actuating means 4 when they are operated.

In the preferred, non-limiting embodiment illustrated herein, there is a thread 18 on the inside of the tubular body 2, coinciding with the end 2b, on which a screw 19 engages for the purpose of juxtaposing the two stumps of fractured bone, as explained in more detail later on, and a thread 20 coinciding with the central portion of the tubular body 2 for coupling with a corresponding thread belonging, as we shall see better later on, to parts of the actuating means 4.

The nail 1 also comprises a cap 21, shown in FIGS. 1, 1a and 2 with an axial hole 22 and a single or double arched appendage 23, suitable for resting against the outer wall of the bone.

The cap 21 is applied to the tubular body 2, engaging with a screw 19 in the thread 18 at the end 2b of the tubular body 2, through the axial hole 22 in said cap 21, enabling the proximal fixing of the nail 1 to the bone and the simultaneous compacting of the fractured stumps of bone.

The cap 21 also comprises a sleeve 24 with an octagonal cross-section and integral at its upper end with the appendage 23, designed to engage in the octagonally-shaped stretch of the end 2b.

This advantageously enables any accidental rotation of the fractured bone stumps to be prevented during the time it takes to reduce the fracture, since the above-described coupling between the cap 21 and the tubular body 2 prevents any mutual rotation between said two elements.

Moreover, said coupling advantageously enables a dynamic compacting of the fractured bone stumps, which are submitted to an axial load in that the cap 21 is capable of sliding on the end 2b of the tubular body 2 during the consolidation of the fractured bone.

It is worth noting that the body 2 advantageously had at least one through hole 90 lying crosswise and, more in particular, orthogonally to the longitudinal axis 29 of the body 2, that is visible, for instance, in FIGS. 1, 1a and 2, each hole being designed to contain a corresponding anchoring screw 91, also called a cross screw, that can be used either in combination with the cap 21 or alone to achieve the proximal anchoring of the device 1.

In other words, according to a preferred embodiment, the cap can be substituted by the above-mentioned cross screw 91.

Said cross screw is particularly useful in the case of multi-fragmentary fractures.

It should also be noted that said at least one through hole may have any shape of profile, and in particular it may be circular and/or elliptic, and/or polygonal, and/or mixtilinear, and may also be designed, as explained later on, to contain another device according to the intervention.

It is also worth noting that, in other embodiments of the present invention destined for use in reducing other types of fracture, the main body 2 may extend axially in a slightly arched conformation, or it may have a non-circular cross-section, such as a polygon, for instance, an ellipse or a mixtilinear cross-section. More in general, the shape of the body 2 substantially reproduces the shape in the longitudinal direction of the bone to which it is applied.

In the non-limiting embodiment illustrated herein, the through openings 5 are provided near the lower, or distal end 2a of the tubular body 2, to enable the distal attachment of the nail 1 to the inside wall of the bone, and they are preferably in the shape of a slot, as shown in FIGS. 8 and 9.

It should be noted that, in the preferred embodiment illustrated herein, the walls of the opening 5 (through which the pins 3 move when they are actuated by the actuating means 4 in order to make them engage in the bone) preferably form an acute angle B with respect to the longitudinal axis 29 of the nail 1. More in particular, they tilt towards the upper, proximal end 2b and they form an angle preferably coming between 15° and 35° with the longitudinal axis 29 of the tubular body 2. Said type of pin 3 is also defined as everted.

More precisely, in the particular embodiment also illustrated in FIG. 9, the axis 55 of the openings 5 forms an angle B of 30° with the axis 9.

In another embodiment said angle B comes between 72° and 68°.

Other good result are reached with an angle B of 35°.

Other good result are reached with an angle B comes between 145° and 165°, and that preferably amounts to 150°.

Combined with the particular shape and size of the pins 3, and with the aforesaid elastic force, said solution also advantageously helps to facilitate the emergence of the pins from the corresponding openings 5 and the consequent anchoring of the pins 3 in the wall of the bone.

Said tilting of the wall 51 of the openings 5, visible in detail in the enlargement in FIGS. 9, also advantageously enables the pins 3 to reach and penetrate the wall of the bone at a coupling angle designed to ensure a perfect, stable grip, with no movements or unwanted displacements.

This result is also assured by the particular conformation and curvature of the pins 3 and of their tips 8.

It should be noted that, in the preferred embodiment of the invention illustrated herein, the walls 52 of the openings 5 (through which the pins 3 move when they are actuated by the actuating means 4 to withdraw them from the bone), i.e. the walls closer to the proximal end, are also tilted with respect to the longitudinal axis 29 of the nail 1; in particular, they form an angle C with the longitudinal axis 29 of the tubular body 2 that comes between 145° and 165°, and that preferably amounts to 150°.

In another embodiment said walls 51, 52 form an angle with respect to the longitudinal axis 29 of said main body 2 that comes between 72° and 68°.

In another embodiment said walls 51, 52 form an angle with respect to the longitudinal axis 29 of said main body 2 that comes between 15° and 35°.

Other good result are reached with an angle of 35°.

This advantageously facilitates the withdrawal of the pins 3 inside their respective seats 6, when the surgeon removes the nail 1. Said solution also enables each pin 3 to slide substantially over the same trajectory both during their anchoring to the bone and during their withdrawal.

It should be noted that, in other embodiments of the present invention, the openings 5 may have different tilting angles providing they are suitable for achieving the previously-stated aims of the invention.

The actuating means 4, visible in FIGS. 1, 1a and 2, are arranged inside the tubular body 2, and comprise a first element 10 co-operating with a second element 15 (also called a stud), shown respectively in FIGS. 10, 11 and 12, 13.

More in particular, in the preferred, non-limiting embodiment, the first element 10 is substantially cylindrical and designed to slide axially inside the main body 2.

It identifies a part of the seats 6 and is complete with a threaded axial hole 12 at one end, and a notch 13 at the opposite end, that is designed to contain the tip of a tool, which preferably consists of a screwdriver. During the assembly of the nail, the operator can take action, by suitably manoeuvring this screwdriver, to rotate and properly orient the element 10 so that the tips 8 of the pins 3 engage in the corresponding openings 5.

This advantageously enables the cylindrical element 10 to slide inside the tubular body 2 along the longitudinal axial 29, while preventing it from rotating around said axis.

The second element, or stud, 15 has a first end 15a and a second end 15b.

The first end 15a is complete with a first, preferably rightward-turning thread, designed to engage in the central threaded portion 20 of the tubular body 2.

The second end 15b is complete with a second thread turning in the opposite direction to the first and suitable for engaging in the threaded hole 12 in the cylindrical element 10.

In line with the end 15a, the stud 15 is also complete with an axial hole 16 that has a substantially hexagonal cross-section designed to contain the corresponding end of a tool preferably consisting of a long, slender hexagon-head wrench T, shown in FIG. 23, that is used by the surgeon—as explained in more detail later on—to take action on the actuating means 4.

More in particular, by taking action on said wrench T as shown in FIG. 24, the surgeon is able to make the stud 15 rotate and thereby obtain a displacement of the cylindrical element 10 along the longitudinal axis 9, in one direction or the other, depending on the sense of rotation of the stud 15.

More precisely, by turning the stud 15 anticlockwise, the surgeon determines a displacement of the stud, and the consequent entrainment of the cylindrical element 10, towards the upper end 2b of the tubular body 2, thereby inducing the emergence of the pins 3.

Vice versa, by turning the stud 15 in a clockwise direction, the surgeon makes the pins 3 withdraw into their respective seats 6.

As concerns the seats 6, it should be noted that they are created longitudinally on the outer surface of the cylindrical element 10.

To be more precise, they comprise a first portion 6a designed to contain the end 7b of the corresponding pin 3, and a second portion 6b suitable for containing the remainder of the pin 3.

It should be noted, in particular, that the cavity 6b has a depth P, or height, that is less than the length, or height h, of the pin 3 that it contains, and this guarantees that the pin 3 is compressed when located inside the seat 6.

More in particular, said depth P preferably comes between 2.2 mm and 3 mm, and amounts to 2.2 mm in the particular embodiment illustrated herein.

It should be noted that said depth may have a different dimension in other embodiments of the invention, providing that it is suitable for achieving the previously-stated aims of the invention.

More in general, said depth depends not only on the height h of the pin that it must contain, but also on the elastic characteristics of said pin.

It should also be noted that, in the non-limiting embodiment of the invention illustrated herein, the number of seats 6 coincides with the number of pins 3, and more in particular it amounts to a number of three. The seats 6 are also arranged at equidistant angles around the longitudinal axis 11 of the cylindrical element 10, at an angle D of 120°, as shown in FIG. 8.

Clearly, in other embodiments of the present invention, there may also be a different number of seats 6, providing it coincides with the number of the pins 3, and of the openings 5, and these may be suitably arranged, providing they can achieve the previously-stated aims of the present invention.

Another variant of the invention differs from the previously-described embodiment in that each anchoring element 3 is moved into its second conformation by the action of members that take effect by means of direct contact with said element. More in particular, in a first solution, said members comprise a sloping plane against which each element comes into contact when the surgeon uses the actuating means 4.

Another variant of the invention differs from the previously-described embodiment in that the elastic means preferably comprise one or more springs that have one end in contact with a wall of the seat 6 and the other abutting against the pin 3.

Another variant of the invention differs from the previously-described embodiment in that the proximal fixing of the nail 1 is achieved by means of the cross screw 91, which engages in the crosswise hole 90 in the device 1.

Said embodiment is particularly suitable for use in the case of multifragmentary fractures, to prevent the displacement of the device due to muscle contraction and the consequent disruption of the fracture.

Another embodiment of the present invention, identified globally by the numeral 100 in FIGS. 14 and 15, differs from the previous embodiments in that the pins are arranged so as to present their concavities facing in the opposite direction to the previously-described solutions, i.e. with their concavities not facing the axis of the nail 1 (also called inverted pins).

In this case, the walls of the openings 5 slope in the opposite direction to those of the previous cases, i.e. they slope towards the so-called distal end 2a. Said walls are designed to contain the tip 8 of the corresponding shank 7 of the pin that, in this case, faces towards the lower end 2a of the main body 2.

It should be noted that this type of pin is particularly suitable for treating simple or multifragmentary fractures occurring in the vicinity of the distal portion of the bone O.

Another embodiment of the present invention, identified globally by the numeral 200 in FIG. 16, is distinguishable from the previously described embodiments in that it comprises two opposite sets of anchoring devices 3, with a space between them, along the longitudinal axis 29, with their respective tips 8 facing one another and oriented in a convergent direction with respect to the axis 29.

The anchoring elements 3 can each be actuated by a corresponding stud element 15. According to a preferred embodiment, the two elements 15 are also mechanically connected to one another to enable the surgeon to actuate the anchoring elements 3 simultaneously.

In this embodiment, the nail is said to be “double-acting” and it is particularly suitable for the treatment of multifragmentary fractures.

Another embodiment of the invention, identified globally by the numeral 300 in FIG. 17, differs again from the previously-described embodiments in that the anchoring elements 3 are arranged with their respective tips 8 not facing one another and oriented in a divergent direction with respect to the axis 29.

Another embodiment of the present invention, identified globally by the numeral 400 in FIG. 18, differs again from the previously-described embodiments in that it comprises two devices of the above-described type that cooperate with one another, indicated respectively by the numerals 500 and 600.

Said device 400 is particularly suitable for use in the treatment of the so-called peri-/sub-trochanteric fractures.

More in particular, this embodiment differs from those previously described in that at least a portion of the external profile of the device 600, shown in particular in FIG. 19, is polygonal or, more in general, mixtilinear. To be more precise, said portion coincides with the proximal end of the device 600 and advantageously enables the coupling of two devices, 500 and 600, preventing their mutual rotation in relation to their corresponding longitudinal axes.

It should be noted, moreover, that this last solution involves the use of a device 500 with a tubular body 502 that extends axially in a slightly curved shape designed to reflect the shape of the bone, and that it involves the insertion of a further device 600 designed to adhere, by means of the pins 3, to a part of the bone substantially at a far from negligible distance from the fracture.

It should also be noted that, in this case, the fracture is reduced by using two devices, one with everted pins 3 and the other with inverted pins 3.

It should further be noted that, in the above-mentioned solution, the device 402 prevents any rotation of the proximal part of the fracture, while any rotation between the two devices 500 and 600 is prevented by the presence of an octagonally-shaped connection, shown in FIG. 19.

In another embodiment of the invention, shown in FIG. 20, a nail 700 is inserted in a fractured neck of femur.

It should be noted that the anchoring elements 3 of the device 700 are positioned in line with the part of the femur 81 coinciding with its maximum diameter. Said anchoring elements 3 have their tips 8 arranged towards the distal end 702a of the tubular body of the device 700.

The end 702b of the tubular body 702 is fixed to the stump 82 of the fractured bone by means of a screw 701, also shown in FIGS. 21 and 22, which is first inserted through 30 a special sleeve 724 in the shape of an expansion washer with two symmetrical lips 723, then screwed onto the end 702b of the tubular body 702 of the nail 700.

Below is a description of how the nail of the invention functions, given with reference to the preferred embodiment illustrated in FIG. 1, there being no substantial differences with respect to the other embodiments described.

After making a hole in the patient's bone that needs to be reduced, the surgeon inserts the nail 1, configured as shown in FIG. 24, inside the medullary canal.

More in particular, the nail is configured with all the elements 3 contained and compressed in their respective seats 6, with their tips engaging in their respective openings 5, but not extend from the body 2, as shown in the enlargement in FIG. 24a.

The surgeon then takes action with the wrench to rotate the stud 15.

This makes the elements 3 move axially so that the point of contact between the surface 31 of each element 3 and the edge 50 of the opening 5 is displaced, thereby allowing each element 3 to return elastically to its natural shape.

In fact, the tip 8 of each pin 3 will naturally tend to move and emerge from the body 2, moving towards the wall of the bone, as shown in particular in FIG. 24b.

This solution advantageously prevents the anchoring elements 3, and their tips in particular, from coming into contact with the edge 52 of the opening 5 during the emergence of the pins 3 through their respective openings. In fact, the aforesaid elastic force drives the end of the pin to engage with the bone, facilitating said action.

By continuing to take action on the wrench T, the surgeon ensures that the pins 3 extend further outwards, moving them along the longitudinal axis 29 of the device 1 until a rotation of each pin 3 is obtained with respect to the axis 30 lying crosswise to the longitudinal axis 29 of the device 1.

Moreover, the surface 32 of each pin 3 comes into contact with the other wall of the opening 5, identified by the numeral 52. This advantageously guides the pin 3 along its trajectory to become implanted in the cortical layer of the bone, exploiting the elastic features of the pin.

This advantageously facilitates the emergence of the pins 3.

It also enables the angle of incidence between each pin and the cortical bone to be controlled, thereby improving the efficacy of the connection.

Finally, after anchoring the pins 3, the surgeon removes the wrench T and installs the cap 21 or cross screw 91, fixing it to the bone.

To remove the device, the surgeon completes the above-described procedure in reverse order. In this case, the elastic action facilitates the withdrawal of each pin 3.

In other words, each anchoring element 3 is designed to move elastically from a first position at rest into a second working position, also facilitated in doing so by its rotation around the crosswise axis 30.

The natural tendency of each anchoring element to return elastically to its original shape facilitates both the emergence from and its withdrawal inside the device 1.

In fact, as soon as its tip reaches the opening 5, the pin tends naturally to regain its original shape, so the tip 8 extends from the main body 2 and, in particular, it goes beyond the thickness of the edge of the main body surrounding the opening 5. This ensures that the anchoring element 3 emerges from its seat 6 without its tip 8 being able to reach the opposite edge 52 of the opening, and consequently risk interfering with the emergence of the pin 3.

The elements 3 thus emerge smoothly and slidingly, in a radial direction, from the tubular body 2 through their corresponding openings 5 to become implanted in the inside wall P of the bone O, without catching and/or seizing, and/or encountering any obstacles, and/or anything else that might prevent or restrict this action.

This also advantageously enables the pins 3 to slide and slip effectively through the openings 5 to withdraw inside their seats 6 when the surgeon takes action on the actuating means 4 during the removal of the nail 1.

It should be noted, moreover, that the distal fixing of the device achieved by means of the pins that become anchored to the fractured bone stump makes it unnecessary to take any x-rays to identify the hole for containing the fixing cross screw, as in the case of the nails of conventional type.

Although the invention has been described with reference to the attached drawings, it may undergo modifications during the implementation stage, all forming part of the same inventive concept expressed by the following claims and consequently equally protected by the present patent.

It is also emphasised that, where the characteristics mentioned in the following claims are followed by reference signs, these are given merely to facilitate the reader and shall not be intended as limiting the interpretation of said claims in any way.

It is also emphasised that all the details of the invention may be replaced by other technically equivalent elements, and that the materials used may be any, according to need, providing they are compatible with the contingent usage, and the same applies to the dimensions of the various elements.

1.-32. (canceled)

33. A device for reducing a bone fracture (1, 100, 200, 300, 400, 500, 600, 700) comprising a main body (2) and one or more anchoring elements (3) designed to be displaced by actuating means (4) from a position at rest inside respective seats (6) of said main body (2) to a working position outside said main body (2), wherein said one or more anchoring elements (3) are pushed outside said respective seats (6) of said main body (2) through a corresponding opening (5) when they are activated by said actuating means (4); characterized in that said anchoring elements (3) are made of an elastic material, and in that said seats (6) comprise a portion (6b) suited to contain a part of a corresponding anchoring elements (3), said portion (6b) having a depth (P), or height, that is less than the length, or height (h), of said corresponding anchoring elements (3) so that in said position at rest, each of said one or more anchoring elements (3) is compressed inside its corresponding seat (6) so as to acquire a first shape; and in that said anchoring elements (3) acquire a second shape differing from said first shape when they are moved to said working position outside said body (2).

34. A device according to claim 33, characterized in that, in said position at rest, each of said one or more anchoring elements (3) is arranged so that its end designed to become implanted in the bone (O) enters said opening (5) but does not extend from said main body (2).

35. A device (200, 300) according to claim 33, characterized in that, during the emergence/withdrawal of said one or more anchoring elements (3) from/in said seat (6), a surface (31, 32) of said anchoring element (3) comes into contact with a wall (50, 51, 52) of said opening (5) to guide each of said one or more anchoring elements (3).

36. A device (200, 300) according to claim 33, characterized in that, during the emergence of said one or more anchoring elements (3), they each rotate around an axis (30) lying crosswise to the longitudinal axis (29) of said device (1) to facilitate their emergence through said at least one opening (5).

37. A device according to claim 33, characterized in that said anchoring elements (3) comprise a shank (7) with a first end (7a) with a pointed terminal portion (48) suitable for becoming implanted in the bone, and a second end (7b) complete with means (9) for connecting said actuating means (4), said shank being shaped and having a length (L) and a height (h).

38. A device according to claim 37, characterized in that said shaped shank (7) is arched and substantially reproduces an arc of a circle, or an arc of an ellipse, or an arc of a parabola.

39. A device according to claims 37, characterized in that said means of connection (9) consist of one end (7b) of each anchoring element (3), said end being suitably shaped so as to fit inside a corresponding cavity (8) in said actuating means (4).

40. A device according to claim 33, characterized in that said actuating means (4) comprise a first element (10) designed to slide axially inside said main body (2) mechanically connected to a second element (15) with a first thread suitable for coupling with a corresponding thread provided in said main body (2), said second element (15) being designed to be rotated by means of a tool (T).

41. A device according to claim 40, characterized in that at least a part of said seats (6) are created longitudinally on the outer surface of said first element (10) and comprise a first portion (6a) suitable for housing one end (7b) of said at least one anchoring element (3).

42. A device according to claim 41, characterized in that said portion (6a) of said seat (6) has a depth (P) that is less than the height (h) of the anchoring element (3) that it contains.

43. A device according to claim 33, characterized in that said openings (5) are slots surrounded by walls (51, 52).

44. A device according to claim 43, characterized in that said walls (51, 52) form an angle with respect to the longitudinal axis (29) of said main body (2) that comes between 72° and 68°.

45. A device according to claim 44, characterized in that said walls (51, 52) form an angle with respect to the longitudinal axis (29) of said main body (2) amounts to 35°.

46. A device according to claim 33, characterized in that the axis (55) of said openings (5) preferably forms an acute angle (B) with respect to the longitudinal axis (29) of said main body (2).

47. A device according to claim 46, characterized in that said angle (B) comes between 72° and 68°.

48. A device according to claim 46, characterized in that said angle (B) amounts to 35°.

49. A device according to claim 46, characterized in that said angle (B) comes between 15° and 35° and preferably amounts to 30°.

50. A device according to claim 33, characterized in that said one or more anchoring elements (3) are arranged radially at an angle (A) amounting to approximately 120° from one another with respect to the longitudinal axis (9) of said main body (2).

51. A device according to claim 33, characterized in that said main body (2) comprises a hollow body with said openings (5) and with a first and a second end (2a, 2b).

52. A device according to claim 33, characterized in that said main body (2) has at least one stretch with a substantially polygonal external shape.

53. A device according to claim 33, characterized in that it also comprises a cap (21) suitable for being placed on the external wall of the bone and anchorable to said bone.

54. A device according to claim 33, characterized in that it also comprises a crosswise through hole (90) suitable for containing a screw (91) for anchoring to said bone and/or another device (1).

55. A device according to claim 54, characterized in that said crosswise through hole (90) has a circular and/or elliptic, and/or polygonal, and/or mixtilinear cross-section.

56. A device according to claim 47, characterized in that said walls (51, 52) form an angle with respect to the longitudinal axis (29) of said main body (2) that comes between 145° and 165°, and preferably amounts to 150°.
